(19) United States
(12) Patent Application Publication
(10) Pub. No.: US 2020/0247831 A1
(43) Pub. Date: Aug. 6, 2020

(54) AMINO-SILA-PYRONIN COMPOUND-BASED ONE- OR TWO-PHOTON ABSORPTION FLUORESCENT SUBSTANCE AND USE THEREOF

(71) Applicant: POSTECH ACADEMY-INDUSTRY FOUNDATION, Pohang-si (KR)

(72) Inventors: Kyo Han AHN, Pohang-si (KR); Hye Gun RYU, Daegu (KR); Kyeong Hwan KIM, Gyeongsan-si (KR); Yong Woong JUN, Pohang-si (KR); Subhankar SINGHA, Pohang-si (KR)

(21) Appl. No.: 16/642,439

(22) PCT Filed: Aug. 31, 2018
(86) PCT No.: PCT/KR2018/010154

§ 371 Date: Feb. 27, 2020

(30) Foreign Application Priority Data
Aug. 31, 2017 (KR) 10-2017-0110708
Aug. 30, 2018 (KR) 10-2018-0102776

(51) Int. Cl. C07F 7/08; A61K 49/00

(52) U.S. Cl. CPC C07F 7/081; A61K 49/0017

(57) ABSTRACT

The present invention relates to a silicon-substituted amino-pyronin compound derivative, a cell- or tissue-imaging method using the same, a cancer diagnosis method using the same, and a method for preparation of the same. More specifically, serving as a two-photon absorption fluorescent substance which has longer absorption and emission wavelengths in red or near-infrared regions of 625 nm or greater, compared to conventional one-photon absorption fluorescent substances, the silicon-substituted amino-pyronin compound derivative can minimize the influence of self-fluorescence in a bioimaging study and is thus expected to be suitable for high-resolution imaging in deep tissues. A fluorescent probe developed on the basis of the fluorescent substance platform is expected to find an expanded range of applications in analyzing and imaging specific materials in vivo.

### TECHNICAL FIELD

The present invention relates to an amino-Si-pyronin compound-based one- or two-photon absorption fluorescent substance and a use thereof, and more particularly, to an application of the fluorescent substance to cell imaging.

### BACKGROUND ART

Bioimaging techniques based on fluorescent signals have been widely utilized as a method capable of visualizing cell organelles, tissues, and the like, and among them, the development of a fluorescent probe which emits fluorescent signals extends the range of use in analyzing and imaging a specific substance in an organism. Most fluorescent materials and probes currently utilized in bioimaging are one-photon absorption fluorescent substances, and bioimaging is realized by one-photon microscopy (OPM).

However, recently, there has been a growing interest in studies on bioimaging using two-photon microscopy (TPM) which enables high-resolution imaging of even deeper tissues without being sensitive to the effects of light scattering. Since a two-photon microscopy imaging method enables excitation in the near-infrared (NIR) regions close to a wavelength band of 900 nm and simultaneously can excite only the focal point portion along with high tissue permeability, the two-photon microscopy imaging method has various advantages in the field of bioimaging such as low photo-damage to biological tissues and low photo-bleaching for fluorescent substances.

Representative examples of a two-photon absorption fluorescent substance for bioimaging using two-photon microscopy include acedan, naphthalimide derivatives, and the like. These fluorescent substances usually emit fluorescence in the green region, and this region overlaps with a self-fluorescence region emitted from innate biomolecules, and thus has a disadvantage in that the reliability of the tissue imaging result is reduced. Accordingly, when a new fluorescent substance which emits strong fluorescence even in long wavelength regions of 625 nm or greater (red or near-infrared rays) beyond the self-fluorescence region due to biomolecules is developed, the new fluorescent substance is expected to overcome the limitations that existing two-photon absorption materials have.

Meanwhile, the greater the difference (Stokes shift) between the excitation wavelength and the emission wavelength of the fluorescent probe is, the greater the decrease in the amount of quenching due to the self-absorption of the fluorescent material or probe can be. Further, since a fluorescent probe which can be excited at different wavelength regions and simultaneously emits at different wavelength regions can obtain a ratiometric fluorescent signal and can overcome characteristics that the fluorescence intensity very sensitively responds depending on the external environment, a reliable quantitative analysis is possible. Accordingly, the fluorescent probe having characteristics as described above is very useful for sensing a specific substrate in an organism and for bioimaging studies (Korean Patent No. 10-1662427).

Therefore, the development of a fluorescent probe which emits strong fluorescence even in long wavelength regions and exhibits photophysical properties which absorb and emit at different wavelength regions may be applied widely to various fields of bioimaging by overcoming the existing limitations, and is expected to provide a highly reliable bioimaging analysis technique.

### DISCLOSURE

### Technical Problem

The present invention has been devised to solve the above-described problems in the related art, and an object thereof is to provide an amino Si-pyronin compound-based one- or two-photon absorption fluorescent substance which is a new fluorescent substance compound capable of realizing tissue imaging with high resolution by minimizing the interference of self-fluorescence, a method for preparation of the same, a use of the same, and the like.

However, the technical problems which the present invention intends to solve are not limited to the technical problems which have been mentioned above, and other technical problems which have not been mentioned will be apparently understood by a person with ordinary skill in the art to which the present invention pertains from the following description.

### Technical Solution

The present invention provides an amino Si-pyronin compound represented by the following Chemical Formula 1 or pharmaceutically acceptable salt thereof.

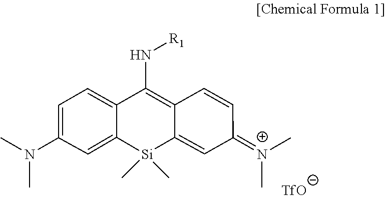

In Chemical Formula 1, R₁ is a substituted or unsubstituted alkyl group having 1 to 18 carbon atoms, the unsubstituted means that a hydrogen atom is substituted with a functional group, and the substituted means that a general functional group is substituted instead of a hydrogen atom. In this case, the functional group is a methyl group, an ethyl group, a phenyl group, an allyl group, a propargyl group, an azidoethyl group, an azidopropyl group, a benzyl group, a tert-butyl butyrate group, an aminobutyl group, a hydroxyethyl group, an aldehyde group, a carboxyl group, a carboxylic ester group, a hydroxyl group, a mercapto group, an azido group, an amine group, a vinyl group, a halogen group, a cyano group, an aryl group, a nitrile group, a carbonate group, and the like, but is not limited thereto.

Further, the present invention provides a julolidine-based amino-Si-pyronin compound represented by the following Chemical Formula 3 or pharmaceutically acceptable salt thereof.

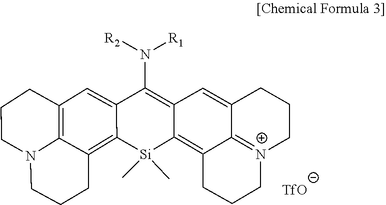

In Chemical Formula 3, R₁ is a substituted or unsubstituted alkyl group having 1 to 18 carbon atoms, the unsubstituted means that a hydrogen atom is substituted with a functional group, and the substituted means that a general functional group is substituted instead of a hydrogen atom. In this case, the functional group is a methyl group, an ethyl group, a phenyl group, an allyl group, a propargyl group, an azidoethyl group, an azidopropyl group, a benzyl group, a tert-butyl butyrate group, an aminobutyl group, a hydroxyethyl group, an aldehyde group, a carboxyl group, a carboxylic ester group, a hydroxyl group, a mercapto group, an azido group, an amine group, a vinyl group, a halogen group, a cyano group, an aryl group, a nitrile group, a carbonate group, and the like, but is not limited thereto.

In addition, R₂ is hydrogen, or a substituted or unsubstituted acyl group, and the unsubstituted means that a hydrogen atom is substituted with a functional group. The substituted means that a functional group is included, and the functional group is a methyl group, an ethyl group, a phenyl group, an allyl group, a propargyl group, an azidoethyl group, an azidopropyl group, a benzyl group, a tert-butyl butyrate group, an aminobutyl group, a hydroxyethyl group, an aldehyde group, a carboxyl group, a carboxylic ester group, a hydroxyl group, a mercapto group, an azido group, an amine group, a vinyl group, a halogen group, a cyano group, an aryl group, a nitrile group, a carbonate group, and the like, but is not limited thereto.

In an embodiment of the present invention, the compound or pharmaceutically acceptable salt thereof may be a one-photon absorption fluorescent substance or a two-photon absorption fluorescent substance, but is not limited thereto as long as the compound or pharmaceutical salt thereof is in a form capable of emitting fluorescence.

In another embodiment of the present invention, the compound or pharmaceutically acceptable salt thereof is preferably a fluorescent substance which emits fluorescence in long wavelength or near-infrared regions of 625 nm or greater, but the region is not limited thereto as long as the region can image cells or tissues.

Furthermore, the present invention provides a cell- or tissue-imaging method, the method including treating cells or tissues with the compound or pharmaceutically acceptable salt thereof and observing the treated cells or tissues.

In an embodiment of the present invention, the observing of the treated cells or tissues is characterized by using a one-photon fluorescence microscope or a two-photon fluorescence microscope.

Further, the present invention provides a composition for diagnosis, including the compound or pharmaceutically acceptable salt thereof.

In addition, the present invention provides a method for obtaining a fluorescence image for diagnosis, the method comprising administering the compound or pharmaceutically acceptable salt thereof in vivo or to a tissue.

Furthermore, the present invention provides a use of the compound or pharmaceutically acceptable salt thereof for diagnosis.

In an embodiment of the present invention, the diagnosis preferably diagnoses a disease such as cancer, tuberculosis, so on. The diagnosis uses the compound present invention or pharmaceutically acceptable salt thereof the alone, or the compound or pharmaceutically acceptable salt thereof bound to a marker of a specific disease, and the disease is not limited thereto.

Further, the present invention provides a method for preparing an amino Si-pyronin represented by the following Chemical Formula 1, the method including: (a) preparing a mixture by adding trifluoromethanesulfonic anhydride to 3,7-bis(dimethylamino)-5,5-dimethyldibenzo[b,e]silin-10(5H)-one; and (b) adding an amine to the mixture.

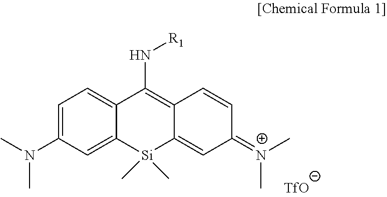

In Chemical Formula 1, R₁ is preferably a substituted or unsubstituted alkyl group having 1 to 18 carbon atoms, but is not limited thereto as long as R₁ has a structure which may be substituted with an amine compound to be added. The amine compound collectively refers to a compound having a form in which a hydrogen atom of ammonia is substituted with a hydrocarbon group.

In addition, the present invention provides a method for preparing a julolidine-based amino-Si-pyronin represented by the following Chemical Formula 2, the method including: (a) preparing a mixture by adding trifluoromethanesulfonic anhydride to a compound represented by the following Chemical Formula 22; and (b) adding an amine to the mixture.

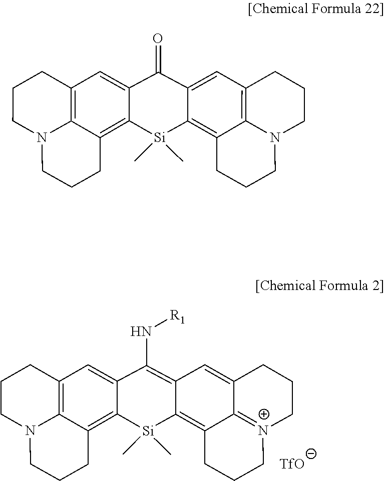

In Chemical Formula 2, R₁ is preferably a substituted or unsubstituted alkyl group having 1 to 18 carbon atoms, but is not limited thereto as long as R₁ has a structure which may be substituted with an amine compound to be added.

Furthermore, the present invention provides a method for preparing a julolidine-based amino-Si-pyronin represented by the following Chemical Formula 3, the method including: (a) preparing a mixture by adding diisopropylethylamine and pyridine to a compound represented by the following Chemical Formula 2; and (b) adding a compound for an acylation reaction to the mixture. The compound for an acylation reaction collectively refers to all compounds which provide an acyl group, and is, for example, a halogenated acyl, a carboxylic anhydride, and the like, but is not limited thereto as long as the compound can provide an acyl group.

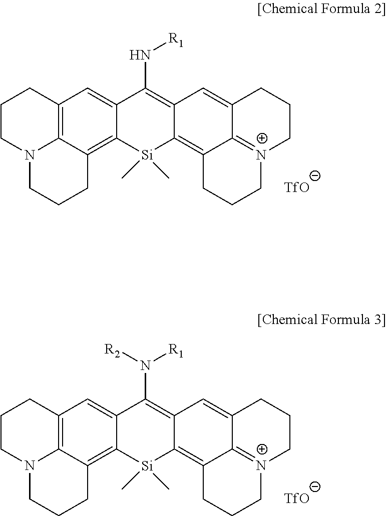

In Chemical Formula 2 and 3, R₁ is preferably a substituted or unsubstituted alkyl group having 1 to 18 carbon atoms, but is not limited thereto, and in Chemical Formula 3, R₂ is preferably a substituted or unsubstituted acyl group, but is not limited thereto.

### Advantageous Effects

Although an amino-Si-pyronin compound-based one- or two-photon absorption fluorescent substance of the present invention is structurally simple, it has absorption and emission wavelengths in red or near-infrared regions, and thus is excellent in chemical and optical stability compared to cyanine-based fluorescent substances which are currently and widely used, and can remarkably enhance the reliability of a test because it is possible to minimize the influence of self-fluorescence in a bioimaging study. Further, when two-photon absorption characteristics of the fluorescent substance of the present invention are used, the fluorescent substance of the present invention can be used even for high-resolution imaging in deep tissues. Therefore, a fluorescent substance platform of the present invention makes it possible to develop a fluorescent probe having various characteristics on the basis of the above characteristics, can be used for imaging and diagnosing not only cancer cells, but also various diseases by using the fluorescent probe alone or binding a target for a marker of a specific disease to the fluorescent probe, and is also expected to remarkably enhance the test accuracy through this. Further, the fluorescent probe is not limited thereto and is expected to have an expanded range of utilization variously applicable to analyzing and imaging specific materials in vivo.

### DESCRIPTION OF DRAWINGS

FIG. 1A is a view illustrating results of confirming the maximum absorption wavelength and maximum emission wavelength values of compounds represented by Chemical Formula 4 to 12 in ethanol, dioxane, dichloromethane, acetonitrile, and a phosphate buffered saline solution according to an example of the present invention.

FIG. 1B is a view illustrating results of confirming the maximum absorption wavelength and maximum emission wavelength values of compounds represented by Chemical Formula 13 to 20 in ethanol, acetonitrile, and a phosphate buffered saline solution according to an example of the present invention.

FIG. 2A is a view illustrating results of confirming fluorescent images obtained from cells using one-photon and two-photon microscopes after cells are treated with compounds represented by Chemical Formula 4 to 12 according to an example of the present invention.

FIG. 2B is a view illustrating results of confirming fluorescent images obtained from cells using one-photon and two-photon microscopes after cells are treated with compounds represented by Chemical Formula 13 to 15 according to an example of the present invention.

FIG. 2C is a view illustrating results of confirming fluorescent images obtained from cells using one-photon and two-photon microscopes after cells are treated with compounds represented by Chemical Formula 16 to 19 according to an example of the present invention.

### MODES OF THE INVENTION

The present inventors have made intensive studies to develop a new fluorescent substance capable of realizing imaging of cells and/or tissues having high reliability and high resolution by minimizing the interference of self-fluorescence of cells or tissues. The fluorescent substance is a fluorescent substance which emits fluorescence in long wavelength regions and near-infrared regions of 625 nm or greater, has the potential to be converted into various derivatives which absorb and emit at different regions as different substitution groups are introduced into an amine group, and thus has an advantage of being suitable for ratiometric bioimaging.

In an example of the present invention, as a result of treating cells or tissues of animal model with the compound of the present invention, it was confirmed that it was possible to exhibit a large absorption cross-sectional value, compared to conventional two-photon absorption fluorescent substances, and to provide excellent fluorescent images of cells. Accordingly, it is possible to provide a cell- or tissue-imaging method using the compound of the present invention or chemically acceptable salt thereof. In addition, it is possible to provide a composition for diagnosis, including the compound of the present invention or pharmaceutically acceptable salt thereof.

In the present specification, the cell or tissue may be a cell or tissue of a mammal including a human or a non-human mammal, and the non-human mammal may include preferably a mouse, a rat, a dog, a cat, a horse, a cow, a sheep, a goat, a rabbit, and the like, but is not limited thereto.

As used herein, an organic solvent collectively refers to a material which dissolves other materials including organic materials, and is preferably ethanol, methanol, toluene, ethylene, phenol, benzene, xylene, dichloromethane, hexane, methyl acetate, ethyl acetate, tetrachloroethylene, and the like, but is not limited thereto as long as the organic solvent can dissolve the compound of the present specification, diisopropylethylamine and pyridine, 2,6-lutidine, diisopropylethylamine, and the like.

As used herein, the compound refers to a pure material including two or more different elements at a predetermined ratio, and may be classified into organic compounds and inorganic compounds according to the inclusion of carbon and hydrogen, and ionic compounds and molecular compounds according to the method of bonding elements, and the compound is preferably one of the compounds represented by Chemical Formulae 1 to 26.

As used herein, the pharmaceutical acceptable salt refers to any organic or inorganic addition salt of the compounds of the present invention, whose effective concentration is non-toxic and harmless and whose side effects caused by the salt do not degrade the beneficial efficacy of the compounds of the present invention. These salts may use an inorganic acid and an organic acid as a free acid, and as the inorganic acid, it is possible to use hydrochloric acid, bromic acid, nitric acid, sulfuric acid, perchloric acid, phosphoric acid, and the like, and as the organic acid, it is possible to use citric acid, acetic acid, lactic acid, maleic acid, fumaric acid, gluconic acid, methanesulfonic acid, glycolic acid, succinic acid, tartaric acid, galacturonic acid, embonic acid, glutamic acid, aspartic acid, oxalic acid, (D) or (L) malic acid, maleic acid, methanesulfonic acid, ethanesulfonic acid, 4-toluenesulfonic acid, salicylic acid, citric acid, benzoic acid, malonic acid, and the like. Furthermore, these salts may include alkali metal salts (sodium salts, potassium salts, and the like), alkaline earth metal salts (calcium salts, magnesium salts, and the like), and the like. For example, an acid addition salt includes an acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methyl sulfate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate, aluminum, arginine, benzathine, calcium, choline, diethylamine, diolamine, glycine, lysine, magnesium, meglumine, olamine, potassium, sodium, tromethamine, zinc salts, and the like, but is not limited thereto as long as the acid addition salt can be added without affecting the efficacy of the compounds of the present invention.

Hereinafter, preferred examples for helping the understanding of the present invention will be suggested. However, the following examples are provided only to more easily understand the present invention, and the contents of the present invention are not limited by the following examples.

### EXAMPLES

### Example 1: Preparation of 3,7-bis(dimethylamino)-5,5-dimethyldibenzo[b,e]silin-10(5H)-one

3,7-Bis(dimethylamino)-5,5-dimethyldibenzo[b,e]silin-10(5H)-one was prepared by a method of the following Reaction Formula 1. Specifically, the compound was prepared by the method of Lukinavicios, G et al., Nat. Chem., 2013, 5, 132-139 and Nagano, T. et al., ACS Chem. Biol., 2011, 6, 600-608.

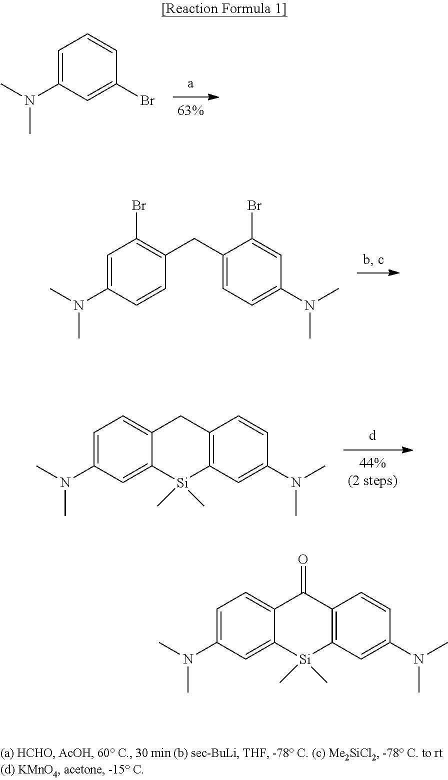

### Example 2: Preparation of Compound Represented by Chemical Formula

A compound represented by Chemical Formula 22 (Compound 22) was prepared by a method of the following Reaction Formula 2.

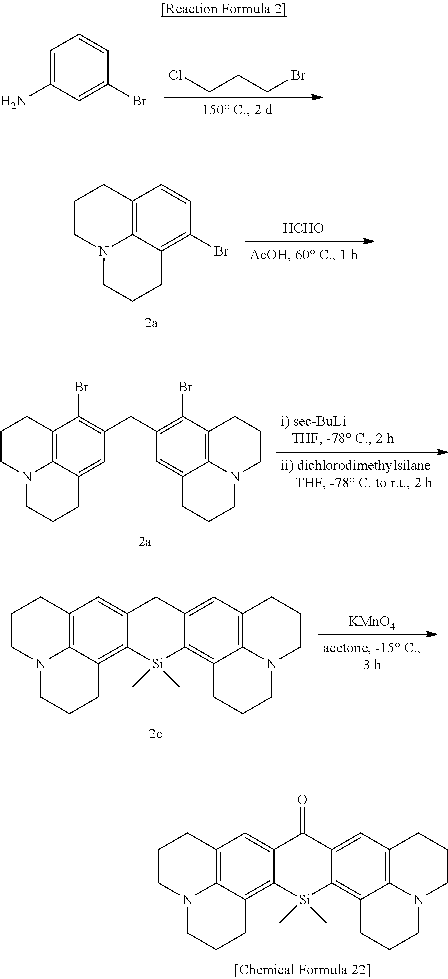

2.1. Preparation of Compound 2a

Compound 2a (8-bromo-1,2,3,5,6,7-hexahydropyrido[3,2,1-ij]quinoline) was prepared by the method of Alexey N. et al., ACS Chem. Biol, 2018, 13, 475-480.

2.2. Preparation of Compound 2b

Compound 2b (bis(8-bromo-1,2,3,5,6,7-hexahydropyrido[3,2,1-ij]quinolin-9-yl)methane) was prepared by the method of Alexey N. et al., ACS Chem. Biol, 2018, 13, 475-480.

2.3. Preparation of Compound 2c

In order to prepare Compound 2c, after a final product (200 mg, 0.39 mmol) obtained in the same manner as in Example 2.2 was dissolved using 15 mL of anhydrous tetrahydrofuran (THF), 0.84 mL of sec-butyllithium (1.4 M) was added thereto at −78° C. And then, after the mixture to which the sec-butyllithium was added was stirred for 2 hours, 85 μL of dimethyldichlorosilane (0.7 mmol) was added thereto. And then, after the mixture was stirred again at room temperature for 3 hours, the reaction was stopped using 1 N hydrogen chloride. Thereafter, after the reaction product was neutralized using saturated sodium hydrogen carbonate, extraction was performed using dichloromethane (2×10 mL) and distilled water, and the organic layer was dried with anhydrous sodium sulfate. And then, after the solvent was completely removed under a reduced pressure condition of 40 mbar, the residue was used in the next step without a separate purification process.

2.4. Preparation of Compound 22

In order to prepare Compound 22, after a final product (160 mg, 0.39 mmol) obtained in the same manner as in Example 2.3 was dissolved using 15 mL of acetone, 185 mg of potassium permanganate (1.17 mmol) was added dropwise thereto at 0° C. for 30 minutes. And then, after the resulting solution was stirred for 3 hours, the solution was diluted using dichloromethane, and then dichloromethane and Celite were mixed and added thereto, and then the solvent was removed under a reduced pressure condition of 40 mbar. And then, the residue was purified using column chromatography in which it passed through silica gel (Merck-Silicagel 60, 230-400 mesh), thereby finally obtaining a yellow solid Compound 22 (33 mg, 20%). During the purification, 3% EtOAc/CH₂Cl₂ was used as an eluent. And then, as a result of observing the prepared Compound 22 by NMR, the NMR spectrum was confirmed as ¹H NMR (CDCl₃, 300 MHz, 298K, δ: 8.07 (m, 2H), 3.29 (d, 8H), 2.9 (m 8H), 2.03 (m, 8H), 0.59 (m, 6H). ¹³C NMR (CDCl₃, 500 MHz, 298K, δ: 143.3, 135.9, 129.6, 128.4, 123.9, 122.6, 50.4, 49.9, 28.7, 28.1, 21.8, 21.5, 0.1.

### Example 3: Preparation of Compound Represented by Chemical Formula 4

A compound represented by Chemical Formula 4 (Compound 4) was prepared by a method of the following Reaction Formula 3.

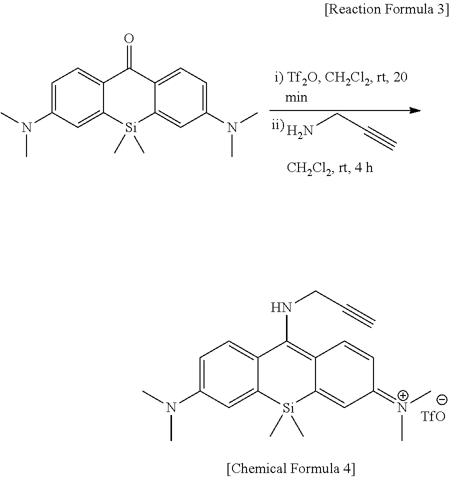

In order to prepare Compound 4, after a final product (100 mg, 0.31 mmol) obtained in the same manner as in Example 1 was dissolved using 3 mL of anhydrous dichloromethane, 104 μL of trifluoromethanesulfonic anhydride (0.62 mmol) was added thereto at room temperature, the resulting mixture was stirred for 20 minutes, and then 0.2 mL of 3.1 mmol propargyl amine was added thereto. And then, after the mixture was stirred again for 4 hours, extraction was performed using dichloromethane (2×10 mL) and distilled water, and the organic layer was dried with anhydrous sodium sulfate. And then, after the solvent was removed under a reduced pressure condition of 40 mbar, the residue was purified using column chromatography in which it passed through silica gel (Merck-Silicagel 60, 230-400 mesh), thereby finally obtaining an orange solid Compound 4 (110 mg, 70%). During the purification, 3% MeOH/CH₂Cl₂ was used as an eluent. And then, as a result of observing the prepared Compound 4 by NMR, the NMR spectrum was confirmed as ¹H NMR (CDCl₃, 300 MHz, 298K, δ: 7.81 (d, 1H), 7.50 (d, 1H), 6.95 (d 1H), 6.91-6.70 (m, 3H), 4.51 (d, 2H), 3.02 (d, 12H), 2.31 (t, 1H), 0.46 (s, 6H). ¹³C NMR (CDCl₃, 500 MHz, 298K, δ: 168.5, 149.9, 149.5, 139.5, 136.5, 135.7, 129.4, 128.2, 127.3, 116.0, 114.6, 113.8, 111.4, 83.1, 70.6, 60.0, 43.5, 40.5, 40.2, 14.2, 0.0, −2.3.

### Example 4: Preparation of Compound Represented by Chemical Formula 5

A compound represented by Chemical Formula 5 (Compound 5) was prepared by a method of the following Reaction Formula 4.

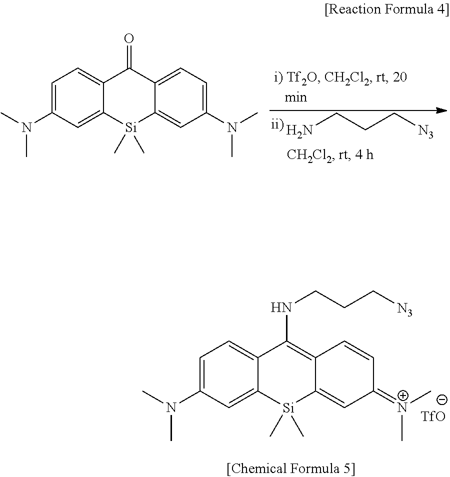

In order to prepare Compound 5, after a final product (100 mg, 0.31 mmol) obtained in the same manner as in Example 1 was dissolved using 3 mL of anhydrous dichloromethane, 104 μL of trifluoromethanesulfonic anhydride (0.62 mmol) was added thereto at room temperature, the resulting mixture was stirred for 20 minutes, and then 310 mg of 3.1 mmol azidopropyl amine was added thereto. And then, after the mixture was stirred again at room temperature for 4 hours, extraction was performed using dichloromethane (2×10 mL) and distilled water, and the organic layer was dried with anhydrous sodium sulfate. And then, after the solvent was removed under a reduced pressure condition of 40 mbar, the residue was purified using column chromatography in which it passed through silica gel (Merck-Silicagel 60, 230-400 mesh), thereby finally obtaining an orange solid Compound 5 (121 mg, 70%). During the purification, 3% MeOH/CH₂Cl₂ was used as an eluent. And then, as a result of observing the prepared Compound 5 by NMR, the NMR spectrum was confirmed as ¹H NMR (CDCl₃, 300 MHz, 298K, δ: 7.81 (d, 1H), 7.38 (d, 1H), 6.98 (d, 1H), 6.87 (d, 1H), 6.83 (dd, 1H), 6.75 (dd, 1H), 3.90 (t, 2H), 3.46 (t, 2H), 3.04 (d, 6.07), 2.08 (q, 2H)), 0.49 (s, 6H). ¹³C NMR (CDCl₃, 500 MHz, 298K, δ: 166.7, 150.0, 149.5, 139.7, 136.4, 136.0, 129.7, 128.0, 127.6, 115.9, 114.9, 113.9, 111.3, 50.8, 49.7, 40.6, 40.3, 31.1, −2.3.

### Example 5: Preparation of Compound Represented by Compound 6

A compound represented by Chemical Formula 6 (Compound 6) was prepared by a method of the following Reaction Formula 5.

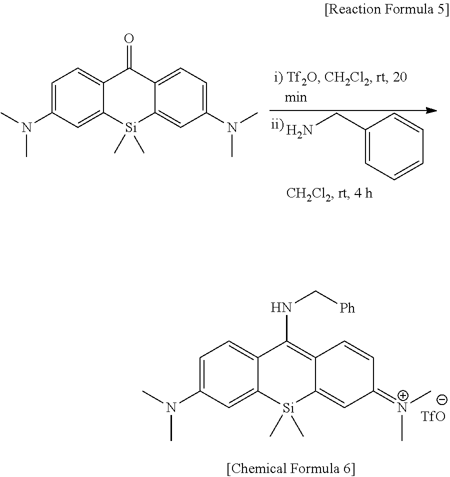

In order to prepare Compound 6, after a final product (100 mg, 0.31 mmol) obtained in the same manner as in Example 1 was dissolved using 3 mL of anhydrous dichloromethane, 104 μL of trifluoromethanesulfonic anhydride (0.62 mmol) was added thereto at room temperature, the resulting mixture was stirred for 20 minutes, and then 332 mg of 3.1 mmol benzyl amine was added thereto. And then, after the mixture was stirred again at room temperature for 4 hours, extraction was performed using dichloromethane (2×10 mL) and distilled water, and the organic layer was dried with anhydrous sodium sulfate. And then, after the solvent was removed under a reduced pressure condition of 40 mbar, the residue was purified using column chromatography in which it passed through silica gel (Merck-Silicagel 60, 230-400 mesh), thereby finally obtaining an orange solid Compound 6 (122 mg, 70%). During the purification, 3% MeOH/CH₂Cl₂ was used as an eluent. And then, as a result of observing the prepared Compound 6 by NMR, the NMR spectrum was confirmed as ¹H NMR (CDCl₃, 300 MHz, 298K, δ: 7.96 (d, 1H), 7.40-7.18 (m, 9H), 6.95 (d, 1H), 6.84 (d, 1H), 6.81 (dd, 1H), 6.65 (dd, 1H), 5.04 (s, 2H), (2.99, 12H), 0.47 (s, 6H). ¹³C NMR (CDCl₃, 500 MHz, 298K, δ: 167.9, 150.2, 149.8, 141.2, 139.9, 136.3, 135.5, 129.7, 128.6 (d), 128.5, 127.6, 127.3, 127.0, 126.8, 126.6, 116.1, 114.9, 113.9, 111.4, 56.8, 40.6, 40.3, −2.2.

### Example 6: Preparation of Compound Represented by Chemical Formula 7

A compound represented by Chemical Formula 7 (Compound 7, mono((2-(1-(3-((7-(dimethylamino)-3-(dimethyliminio)-5,5-dimethyl-3,5-dihydrodibenzo[b,e]silin-10-yl)amino)propyl)-1H-1,2,3-triazol-4-yl)ethyl)triphenylphosphonium)monobromide trifluoromethanesulfonate) was prepared by a method of the following Reaction Formula 6.

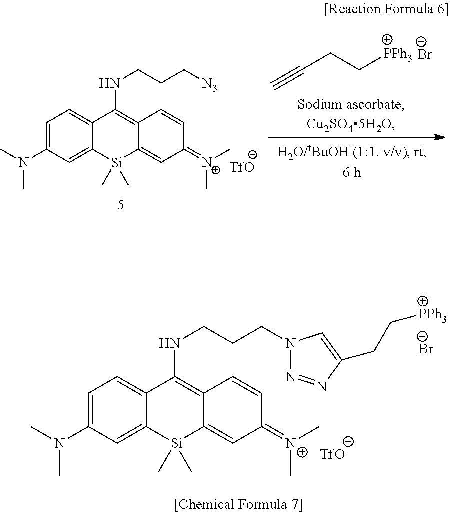

In order to prepare Compound 7, after a final product (56 mg, 0.1 mmol) obtained in the same manner as in Example 4 and but-3-in-1-triphenylphosphonium bromide were dissolved using 3 mL of tertiary butanol and 3 mL of distilled water, 100 μL of sodium ascorbate (1 M) and 2.5 mg of copper sulfate (0.01 mmol) were added thereto, the resulting solution was stirred for 6 hours, and then extraction was performed using dichloromethane (2×10 mL) and distilled water, and the organic layer was dried with anhydrous sodium sulfate. And then, after the solvent was removed under a reduced pressure condition of 40 mbar, the residue was purified using column chromatography in which it passed through silica gel (Merck-Silicagel 60, 230-400 mesh), thereby finally obtaining an orange solid Compound 7 (52 mg, 54%). During the purification, 1% MeOH/CH₂Cl₂ was used as an eluent. And then, as a result of observing the prepared Compound 7 by NMR, the NMR spectrum was confirmed as ¹H NMR (CDCl₃, 300 MHz, 298K, δ: 8.47 (s, 1H), 8.44 (d, 1H), 7.81 (m, 9H), 7.68 (q, 6H), 7.46 (d, 1H), 7.26 (s, 1H), 6.86 (d, 1H), 6.75 (s, 2H), 4.38 (t, 2H), 4.05 (t, 2H), 3.99 (q, 2H), 3.13 (t, 2H), 3.07 (d, 12H), 2.66 (t, 2H), 0.43 (s, 6H). ¹³C NMR (CDCl₃, 500 MHz, 298K, δ: 173.6, 151.9, 151.7, 144.3, 144.2, 142.3, 139.1, 135.4, 135.3, 133.9, 133.8, 131.5, 131.0, 130.7, 124.9, 124.2, 119.9, 118.1, 117.4, 116.4, 115.2, 113.3, 111.9, 47.6, 47.2, 40.1 (d), 29.8, 23.0, 22.6, 19.4 (d), −1.9.

### Example 7: Preparation of Compound Represented by Chemical Formula 8

A compound represented by Chemical Formula 8 (Compound 8, N-(10-((4-(tert-butoxy)-4-oxobutyl)amino)-7-(dimethylamino)-5,5-dimethyldibenzo silin-3(5H)-ylidene)-N-methylmethaneammonium trifluoromethanesulfonate) was prepared by a method of the following Reaction Formula 7.

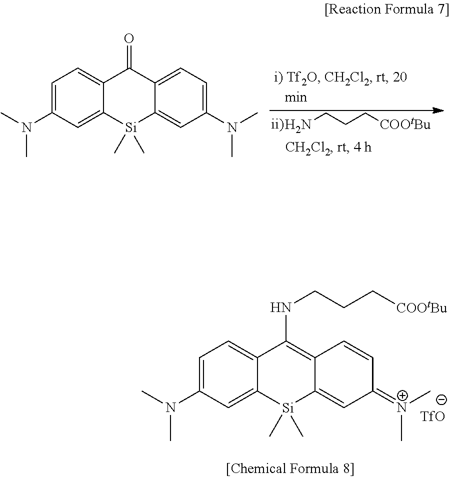

In order to prepare Compound 8, after a final product (100 mg, 0.31 mmol) obtained in the same manner as in Example 1 was dissolved using 3 mL of anhydrous dichloromethane, 104 μL of trifluoromethanesulfonic anhydride (0.62 mmol) was added thereto at room temperature, the resulting mixture was stirred for 20 minutes, and then 493 mg of 3.1 mmol tert-butyl 4-aminobutanoate was added thereto. And then, after the mixture was stirred again at room temperature for 4 hours, extraction was performed using dichloromethane (2×10 mL) and distilled water, and the organic layer was dried with anhydrous sodium sulfate. And then, after the solvent was removed under a reduced pressure condition of 40 mbar, the residue was purified using column chromatography in which it passed through silica gel (Merck-Silicagel 60, 230-400 mesh), thereby finally obtaining an orange solid Compound 8 (121 mg, 70%). During the purification, 3% MeOH/CH₂Cl₂ was used as an eluent. And then, as a result of observing the prepared Compound 8 by NMR, the NMR spectrum was confirmed as ¹H NMR (CDCl₃, 300 MHz, 298K, δ: 8.08 (d, 1H), 7.60 (d, 1H), 6.92 (m, 4H), 4.06 (t, 2H), 3.12 (d, 12H), 2.34 (t, 2H), 2.21 (t, 2H), 1.33 (s, 9H), 0.48 (s, 6H). ¹³C NMR (CDCl₃, 500 MHz, 298K, δ: 173.3, 172.9, 151.9, 151.7, 142.4, 138.8, 132.1, 129.3, 125.0, 119.3, 116.2, 115.2, 113.6, 111.5, 81.2, 50.2, 40.0, 39.9, 32.7, 27.9, 24.2, −0.0, −2.0.

### Example 8: Preparation of Compound Represented by Chemical Formula 9

A compound represented by Chemical Formula 9 (Compound 9, N-(10-((2-((tert-butoxycarbonyl)amino)ethyl)amino)-7-(dimethylamino)-5,5-dimethyldibenzo[b,e]silin-3(5H)-ylidene)-N-methylmethaneammonium trifluoromethanesulfonate) was prepared by a method of the following Reaction Formula 8.

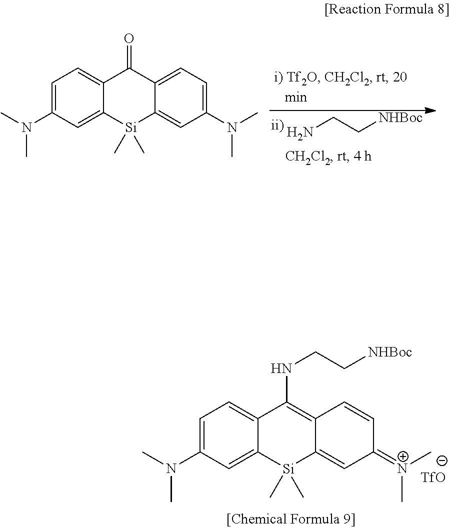

In order to prepare Compound 9, after a final product (100 mg, 0.31 mmol) obtained in the same manner as in Example 1 was dissolved using 3 mL of anhydrous dichloromethane, 104 μL of trifluoromethanesulfonic anhydride (0.62 mmol) was added thereto at room temperature, the resulting mixture was stirred for 10 minutes, and then 496.6 mg of 3.1 mmol tert-butyl(2-aminoethyl)carbamate was added thereto. And then, after the mixture was stirred again at room temperature for 4 hours, extraction was performed using dichloromethane (2×10 mL) and distilled water, and the organic layer was dried with anhydrous sodium sulfate. And then, after the solvent was removed under a reduced pressure condition of 40 mbar, the residue was purified using column chromatography in which it passed through silica gel (Merck-Silicagel 60, 230-400 mesh), thereby finally obtaining an orange solid Compound 9 (134 mg, 70%). During the purification, 3% MeOH/CH₂Cl₂ was used as an eluent. As a result of observing the prepared Compound 9 by NMR, the NMR spectrum was confirmed as ¹H NMR (CDCl₃, 300 MHz, 298K, δ: 7.93 (d, 1H), 7.63 (d, 1H), 6.90 (d, 1H), 6.85 (d, 2H), 6.79 (dd, 1H), 4.14 (t, 2H), 3.66 (q, 4H), 3.14 (d, 12H), 1.36 (s, 9H), 0.48 (s, 6H).

### Example 9: Preparation of Compound Represented by Compound 10

A compound represented by Chemical Formula 10 (Compound 10, N-(10-(but-3-en-1-ylamino)-7-(dimethylamino)-5,5-dimethyldibenzo[b,e]silin-3(5H)-ylidene)-N-methylmethaneammonium trifluoromethanesulfonate) was prepared by a method of the following Reaction Formula 9.

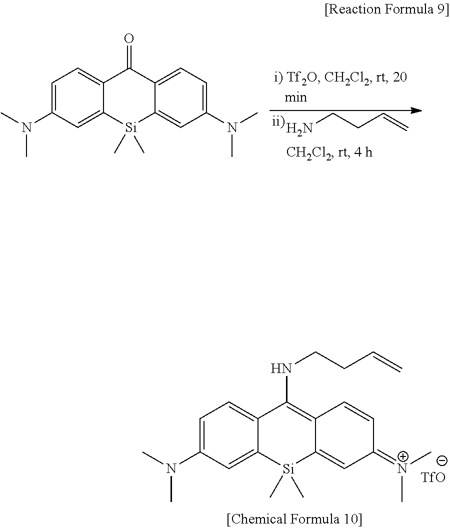

In order to prepare Compound 10, after a final product (100 mg, 0.31 mmol) obtained in the same manner as in Example 1 was dissolved using 3 mL of anhydrous dichloromethane, 104 μL of trifluoromethanesulfonic anhydride (0.62 mmol) was added thereto at room temperature, the resulting mixture was stirred for 10 minutes, and then 220 mg of 3.1 mmol but-3-en-1-amine was added thereto. And then, after the mixture was stirred again at room temperature for 4 hours, extraction was performed using dichloromethane (2×10 mL) and distilled water, and the organic layer was dried with anhydrous sodium sulfate. And then, after the solvent was removed under a reduced pressure condition of 40 mbar, the residue was purified using column chromatography in which it passed through silica gel (Merck-Silicagel 60, 230-400 mesh), thereby finally obtaining an orange solid Compound 10 (114.5 mg, 70%). During the purification, 3% MeOH/CH₂Cl₂ was used as an eluent. And then, as a result of observing the prepared Compound 10 by NMR, the NMR spectrum was confirmed as ¹H NMR (CDCl₃, 300 MHz, 298K, δ: 8.06 (d, 1H), 7.51 (d, 1H), 6.92 (s, 1H), 6.83 (d, 2H), 6.74 (d, 1H), 5.66 (m, 1H), 5.07 (m, 2H), 4.07 (t, 2H), 3.13 (d, 12H), 2.67 (q, 2H), 0.47 (s, 6H). ¹³C NMR (CDCl₃, 500 MHz, 298K, δ: 173.72, 151.8, 151.6, 142.4, 138.7, 133.3, 131.6, 129.4, 125.1, 123.8, 122.0, 119.7, 119.4, 118.4, 116.3, 115.1, 113.5, 111.2, 49.7, 39.9, 33.1, 0.0, −2.2.

### Example 10: Preparation of Compound Represented by Chemical Formula 11

A compound represented by Chemical Formula 11 (Compound 11, N-(10-((3-chloropropyl)amino)-7-(dimethylamino)-5,5-dimethyldibenzo[b,e]silin-3(5H)-ylidene)-N-methylmethaneammonium trifluoromethanesulfonate) was prepared by a method of the following Reaction Formula 10.

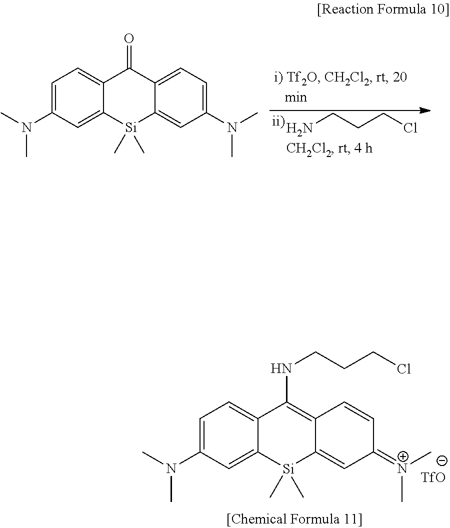

In order to prepare Compound 11, after a final product (100 mg, 0.31 mmol) obtained in the same manner as in Example 1 was dissolved using 3 mL of anhydrous dichloromethane, 104 μL of trifluoromethanesulfonic anhydride (0.62 mmol) was added thereto at room temperature, the resulting mixture was stirred for 10 minutes, and then 290 mg of 3.1 mmol 3-chloropropan-1-amine was added thereto. And then, after the mixture was stirred again at room temperature for 4 hours, extraction was performed using dichloromethane (2×10 mL) and distilled water, and the organic layer was dried with anhydrous sodium sulfate. And then, after the solvent was removed under a reduced pressure condition of 40 mbar, the residue was purified using column chromatography in which it passed through silica gel (Merck-Silicagel 60, 230-400 mesh), thereby finally obtaining an orange solid Compound 11 (119 mg, 70%). During the purification, 3% MeOH/CH₂Cl₂ was used as an eluent. And then, as a result of observing the prepared Compound 11 by NMR, the NMR spectrum was confirmed as ¹H NMR (CDCl₃, 300 MHz, 298K, δ: 7.87 (d, 1H), 7.41 (d, 1H), 6.95 (d, 1H), 6.84 (m, 2H), 6.73 (dd, 1H), 3.99 (t, 2H), 3.69 (t, 2H)), 3.04 (d, 12H), 2.29 (m, 2H), 0.48 (s, 6H). ¹³C NMR (CDCl₃, 500 MHz, 298K, δ: 168.2, 150.3, 149.9, 140.3, 136.5, 134.5, 130.1, 128.4, 126.4, 116.0, 115.0, 113.9, 111.3, 50.2, 43.3, 40.5, 40.3, 34.1, −2.3.

### Example 11: Preparation of Compound Represented by Chemical Formula 12

A compound represented by Chemical Formula 12 (Compound 12, N-(7-(dimethylamino)-5,5-dimethyl-10-((2-morpholinoethyl)amino)dibenzo[b,e]silin-3(5H)-ylidene)-N-methylmethaneammonium trifluoromethanesulfonate) was prepared by a method of the following Reaction Formula 11.

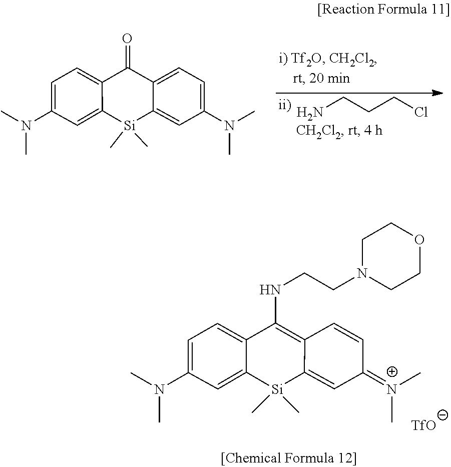

In order to prepare Compound 12, after a final product (100 mg, 0.31 mmol) obtained in the same manner as in Example 1 was dissolved using 3 mL of anhydrous dichloromethane, 104 μL of trifluoromethanesulfonic anhydride (0.62 mmol) was added thereto at room temperature, the resulting mixture was stirred for 10 minutes, and then 403 mg of 3.1 mmol 2-morpholinoethanamine was added thereto. And then, after the mixture was stirred again at room temperature for 4 hours, extraction was performed using dichloromethane (2×10 mL) and distilled water, and the organic layer was dried with anhydrous sodium sulfate. And then, after the solvent was removed under a reduced pressure condition of 40 mbar, the residue was purified using column chromatography in which it passed through silica gel (Merck-Silicagel 60, 230-400 mesh), thereby finally obtaining an orange solid Compound 12 (127 mg, 70%). During the purification, 3% MeOH/CH₂Cl₂ was used as an eluent. And then, as a result of observing the prepared Compound 12 by NMR, the NMR spectrum was confirmed as ¹H NMR (CDCl₃, 300 MHz, 298K, δ: 8.51 (d, 1H), 7.63 (d, 1H), 6.91 (m, 2H), 6.78 (d, 1H), 6.72 (dd, 1H), 4.18 (t, 2H), 3.56 (t, 4H), 3.10 (d, 12H), 2.41 (s, 4H), 0.47 (s, 6H). ¹³C NMR (CDCl₃, 500 MHz, 298K, δ: 173.8, 151.9, 151.5, 142.1, 138.6, 131.3, 131.0, 125.6, 121.0, 116.3, 115.0, 113.5, 111.3, 66.8, 57.0, 53.6, 47.0, 40.1, −1.9.

### Example 12: Preparation of Compound Represented by Compound 13

A compound represented by Chemical Formula 13 (Compound 13) was prepared by a method of the following Reaction Formula 12.

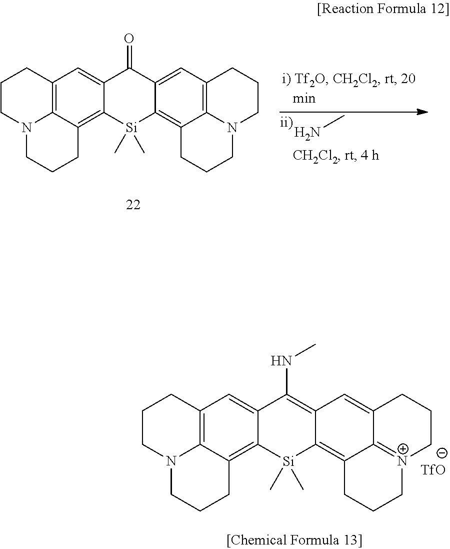

In order to prepare Compound 13, after a final product (70 mg, 0.165 mmol) obtained in the same manner as in Example 2 was dissolved using 3 mL of anhydrous dichloromethane, 57 μL of trifluoromethanesulfonic anhydride (0.62 mmol) was added thereto at room temperature, the resulting mixture was stirred for 20 minutes, and then 0.83 mL of 1.66 mmol methyl amine dissolved with a 2 M tetrahydrofuran solution was added thereto. And then, after the mixture was stirred again at room temperature for 4 hours, extraction was performed using dichloromethane (2×10 mL) and distilled water, and the organic layer was dried with anhydrous sodium sulfate. And then, after the solvent was removed under a reduced pressure condition of 40 mbar, the residue was purified using column chromatography in which it passed through silica gel (Merck-Silicagel 60, 230-400 mesh), thereby finally obtaining an orange solid Compound 13 (68.3 mg, 70%). During the purification, 3% MeOH/CH₂Cl₂ was used as an eluent. And then, as a result of observing the prepared Compound 13 by NMR, the NMR spectrum was confirmed as ¹H NMR (CDCl₃, 300 MHz, 298K, δ: 7.32 (s, 2H), 3.47 (d, 3H)), 3.33 (s, 8H), 2.83 (s, 8H), 2.03 (d, 8H), 0.56 (s, 6H). ¹³C NMR (CDCl₃, 125 MHz, 298 K): d 175.1, 146.6, 129.4, 127.0, 124.7, 122.2, 119.6, 50.7, 50.1, 37.9, 29.9, 29.4, 28.0, 26.7, 21.5, 21.2, 0.2, 0.1,−0.1,−0.3.

### Example 13: Preparation of Compound Represented by Chemical Formula 14

A compound represented by Chemical Formula 14 (Compound 14) was prepared by a method of the following Reaction Formula 13.

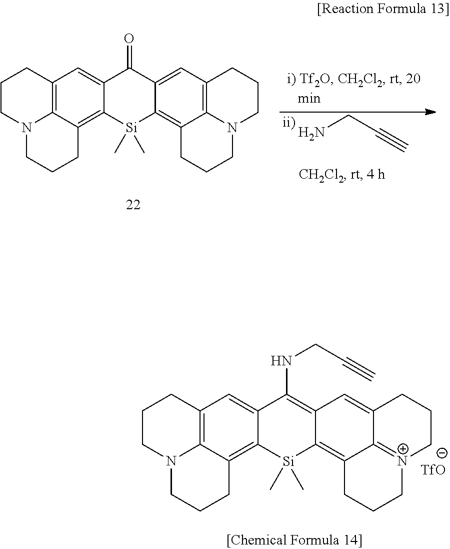

In order to prepare Compound 14, after a final product (35 mg, 0.082 mmol) obtained in the same manner as in Example 2 was dissolved using 2 mL of anhydrous dichloromethane, 27 μL of trifluoromethanesulfonic anhydride (0.16 mmol) was added thereto at room temperature, the resulting mixture was stirred for 20 minutes, and then 52.5 μL of 0.82 mmol propargyl amine was added thereto. And then, after the mixture was stirred again at room temperature for 4 hours, extraction was performed using dichloromethane (2×10 mL) and distilled water, and the organic layer was dried with anhydrous sodium sulfate. And then, after the solvent was removed under a reduced pressure condition of 40 mbar, the residue was purified using column chromatography in which it passed through silica gel (Merck-Silicagel 60, 230-400 mesh), thereby finally obtaining a red solid Compound 14 (35.3 mg, 70%). During the purification, 3% MeOH/CH₂Cl₂ was used as an eluent. And then, as a result of observing the prepared Compound 14 by NMR, the NMR spectrum was confirmed as ¹H NMR (CDCl₃, 300 MHz, 298K, δ: 7.58 (s, 2H), 4.37 (d, 2H), 3.36 (d, 8H), 2.84 (t, 8H), 2.53 (s, 1H), 2.02 (q, 10H), 0.56 (s, 6H). ¹³C NMR (CDCl₃, 125 MHz, 298 K): d 175.4, 147.0, 128.6, 126.5, 123.0, 122.1, 119.6, 78.4, 74.5, 50.8, 50.2, 39.9, 29.9, 29.4, 27.9, 21.5, 21.1, 0.2, −0.1.

### Example 14: Preparation of Compound Represented by Chemical Formula 15

A compound represented by Chemical Formula 15 (Compound 15) was prepared by a method of the following Reaction Formula 14.

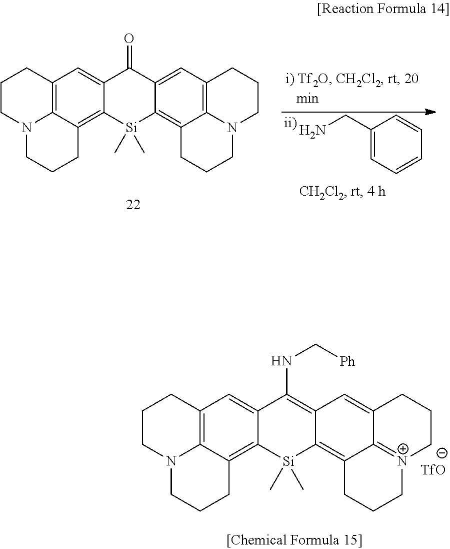

In order to prepare Compound 15, after a final product (20 mg, 0.047 mmol) obtained in the same manner as in Example 2 was dissolved using 2 mL of anhydrous dichloromethane, 16 μL of trifluoromethanesulfonic anhydride (0.093 mmol) was added thereto at room temperature, the resulting mixture was stirred for 20 minutes, and then 51 μL of 0.47 mmol benzyl amine was added thereto. And then, after the mixture was stirred again at room temperature for 4 hours, extraction was performed using dichloromethane (2×10 mL) and distilled water, and the organic layer was dried with anhydrous sodium sulfate. And then, after the solvent was removed under a reduced pressure condition of 40 mbar, the residue was purified using column chromatography in which it passed through silica gel (Merck-Silicagel 60, 230-400 mesh), thereby finally obtaining a red solid Compound 15 (22 mg, 70%). During the purification, 3% MeOH/CH₂Cl₂ was used as an eluent. And then, as a result of observing the prepared Compound 15 by NMR, the NMR spectrum was confirmed as ¹H NMR (CDCl₃, 300 MHz, 298K, δ: 7.39 (m, 7H), 4.91 (s, 2H), 3.35 (q, 8H), 2.85 (t, 8H), 2.00 (m, 8H), 0.56 (s, 6H). ¹³C NMR (CDCl₃, 75 MHz, 298 K): d 176.1, 146.8, 137.3, 129.2, 128.1, 127.7, 126.3, 122.9, 118.7, 53.7, 50.7, 50.2, 29.9, 29.4, 27.9, 21.5, 21.1, 0.2, −0.1.

### Example 15: Preparation of Compound Represented by Compound 16

A compound represented by Chemical Formula 16 (Compound 16) was prepared by a method of the following Reaction Formula 15.

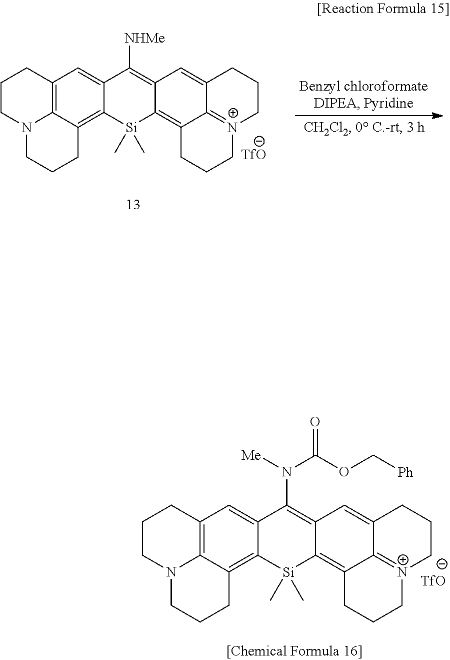

In order to prepare Compound 16, after a final product Compound 13 (30 mg, 0.05 mmol) obtained in the same manner as in Example 12 was dissolved using 5 mL of anhydrous dichloromethane, 44 μL, of 0.25 mmol diisopropylethylamine (DIPEA) and 71 μL of 0.5 mmol benzyl chloroformate were added thereto at 0° C., the resulting mixture was stirred at room temperature for 3 hours, and then extraction was performed using dichloromethane (2×10 mL) and distilled water, and the organic layer was dried with anhydrous sodium sulfate. And then, after the solvent was removed under a reduced pressure condition of 40 mbar, the residue was purified using column chromatography in which it passed through silica gel (Merck-Silicagel 60, 230-400 mesh), thereby obtaining a green solid compound. During the purification, 5% MeOH/CH₂Cl₂ was used as an eluent. And then, Compound 16 (4 mg, 11%) was finally obtained by again purifying the green solid compound through high performance liquid chromatography (HLPC). As a result of observing the prepared Compound 16 by NMR, the NMR spectrum was confirmed as ¹H NMR (CD₃OD, 500 MHz, 298 K): d 7.25-7.21 (m, 3H), 7.10-7.05 (m, 4H), 5.04 (s, 2H), 3.61-3.57 (m, 8H), 3.22 (s, 3H), 2.96-2.94 (m, 4H), 2.72-2.68 (m, 2H), 2.62-2.58 (m, 2H), 2.06-2.05 (m, 4H), 1.97-1.96 (m, 4H), 0.67 (d, J=6.0 Hz, 6H); ¹³C NMR (CD₃OD, 125 MHz, 298 K): d 161.0, 160.5, 157.2, 157.1, 152.0, 142.9, 142.7, 138.3, 137.9, 135.2, 133.4, 129.9, 129.7, 129.6, 129.5, 129.2, 128.9, 126.6, 125.9, 125.7, 69.2, 68.8, 53.1, 52.5, 40.0, 39.8, 33.2, 30.9, 30.6, 30.1, 28.9, 28.8, 26.2, 22.1, 21.9, 14.6,−0.7,−0.8.

### Example 16: Preparation of Compound Represented by Chemical Formula 17

A compound represented by Chemical Formula 17 (Compound 17) was prepared by a method of the following Reaction Formula 16.

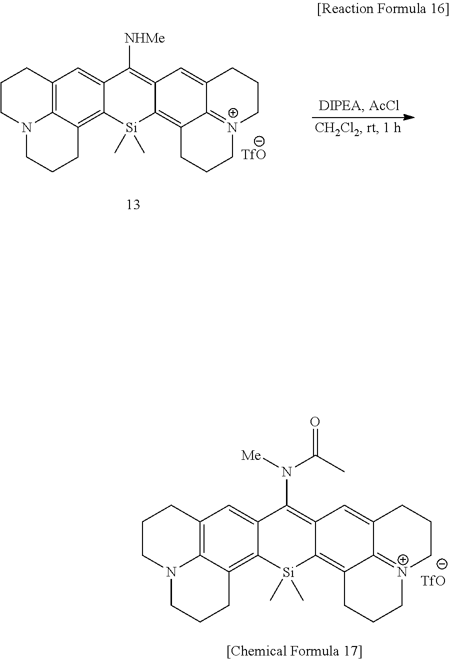

In order to prepare Compound 17, after a final product Compound 13 (30 mg, 0.05 mmol) obtained in the same manner as in Example 12 was dissolved using 5 mL of anhydrous dichloromethane, 87 μL of 0.75 mmol 2,6-lutidine was added thereto at 0° C., the resulting solution was stirred for 10 minutes, and then 36 μL of 0.5 mmol acetyl chloride was added thereto, the resulting solution was stirred at room temperature for 12 hours, and then extraction was performed using dichloromethane (2×10 mL) and distilled water, and the organic layer was dried with anhydrous sodium sulfate. And then, after the solvent was removed under a reduced pressure condition of 40 mbar, the residue was purified using column chromatography in which it passed through silica gel (Merck-Silicagel 60, 230-400 mesh), thereby obtaining a green solid compound. During the purification, 5% MeOH/CH₂Cl₂ was used as an eluent. And then, Compound 17 (5 mg, 15%) was finally obtained by again purifying the green solid compound through high performance liquid chromatography (HLPC). As a result of observing the prepared Compound 17, the NMR spectrum was confirmed as ¹H NMR (CD₃OD, 500 MHz, 298 K): d 7.10 (s, 2H), 3.63-3.60 (m, 8H), 3.22 (s, 3H), 2.98-2.95 (m, 4H), 2.79-2.77 (m, 4H), 2.09-2.06 (m, 4H), 2.03-2.00 (m, 4H), 1.83 (s, 3H), 0.68 (s, 6H); ¹³C NMR (CD₃OD, 125 MHz, 298 K): d 173.0, 160.2, 152.2, 142.6, 134.7, 134.0, 127.2, 125.4, 53.2, 53.1, 52.6, 39.1, 30.1, 28.9, 22.0, 21.9,−0.8.

### Example 17: Preparation of Compound Represented by Chemical Formula 18

A compound represented by Chemical Formula 18 (Compound 18) was prepared by a method of the following Reaction Formula 17.

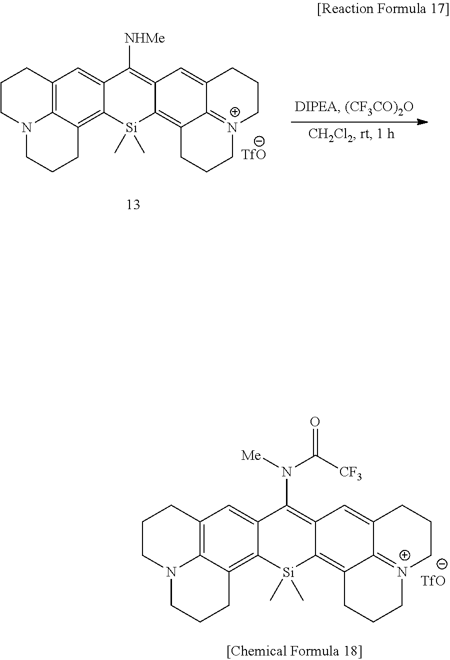

In order to prepare Compound 18, after a final product Compound 13 (30 mg, 0.05 mmol) obtained in the same manner as in Example 12 was dissolved using 10 mL of anhydrous dichloromethane, 44 μL of 0.25 mmol diisopropylethylamine (DIPEA) was added thereto at 0° C., the resulting solution was stirred for 10 minutes, and then 21 μL of 0.5 mmol trifluoroacetic anhydride was added thereto, the resulting solution was stirred at room temperature for 3 hours, and then extraction was performed using dichloromethane (2×10 mL) and 1 N hydrogen chloride, and the organic layer was dried with anhydrous sodium sulfate. And then, after the solvent was removed under a reduced pressure condition of 40 mbar, the residue was purified using column chromatography in which it passed through silica gel (Merck-Silicagel 60, 230-400 mesh), thereby obtaining a green solid compound. During the purification, 5% MeOH/CH₂Cl₂ was used as an eluent. And then, Compound 18 (7 mg, 21%) was finally obtained by again purifying the green solid compound through high performance liquid chromatography (HLPC). As a result of observing the prepared Compound 18 by NMR, the NMR spectrum was confirmed as ¹H NMR (CD₃OD, 500 MHz, 298 K): d 7.08 (s, 2H), 3.64-3.62 (m, 8H), 3.40 (s, 3H), 2.98-2.96 (m, 4H), 2.80-2.76 (m, 4H), 2.09-2.06 (m, 4H), 2.01-1.99 (m, 4H), 0.69 (d, J=15.0 Hz, 6H); ¹³C NMR (CD₃OD, 125 MHz, 298 K): d 156.5, 152.2, 142.2, 134.6, 134.2, 134.0, 133.9, 127.1, 127.1, 125.5, 123.6, 57.6, 53.3, 53.2, 52.6, 41.6, 30.9, 30.1, 28.9, 28.8, 22.0, 21.9, −0.7, −1.0.

### Example 18: Preparation of Compound Represented by Chemical Formula 19

A compound represented by Chemical Formula 19 (Compound 19) was prepared by a method of the following Reaction Formula 18.

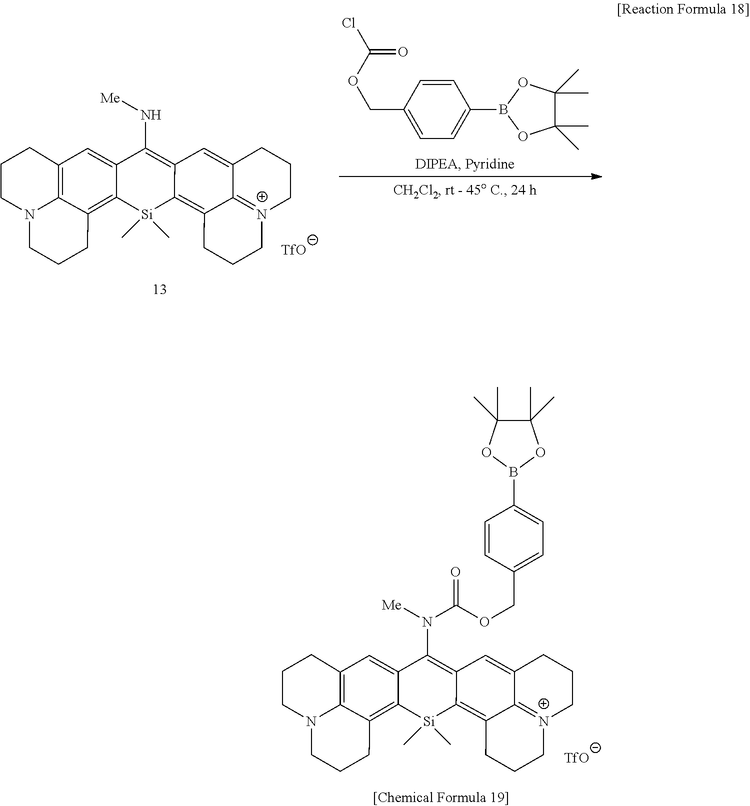 [SMILES: CNC1=c2cc3c4c(c2[Si](C)(C)c2c1cc1c5c2CCCN5CCC1)CCC[N+]=4CCC3 ; CC1(C)OB(c2ccc(COC(=O)Cl)cc2)OC1(C)C ; CN(C(=O)OCc1ccc(B2OC(C)(C)C(C)(C)O2)cc1)C1=c2cc3c4c(c2[Si](C)(C)c2c1cc1c5c2CCCN5CCC1)CCC[N+]=4CCC3]

In order to prepare Compound 19, after a final product Compound 13 (30 mg, 0.05 mmol) obtained in the same manner as in Example 12 was dissolved using 5 mL of anhydrous dichloromethane, 44 μL, of 0.25 mmol diisopropylethylamine (DIPEA) and 40 μL, of 0.5 mmol pyridine were added thereto, the resulting solution was stirred at room temperature for 10 minutes, and then 0.5 mmol 4-(pinacol boronate)-benzylchloroformate was added thereto, and the resulting mixture was mixed by stirring at 45° C. for 24 hours. And then, the mixed solution was extracted using dichloromethane (2×10 mL) and 1 N hydrogen chloride, and the organic layer was dried with anhydrous sodium sulfate. And then, after the solvent was removed under a reduced pressure condition of 40 mbar, the residue was purified using column chromatography in which it passed through silica gel (Merck-Silicagel 60, 230-400 mesh), thereby obtaining a green solid compound. During the purification, 5% MeOH/CH₂Cl₂ was used as an eluent. And then, Compound 19 (3 mg, 8%) was finally obtained by again purifying the green solid compound through high performance liquid chromatography (HLPC). As a result of observing the prepared Compound 19 by NMR, the NMR spectrum was confirmed as ¹H NMR (CDCl₃, 500 MHz, 298 K): d 7.62 (d, J=8.0 Hz, 2H), 7.05 (d, J=10.5 Hz, 2H), 7.00 (s, 2H), 5.09 (s, 2H), 3.64-3.42 (m, 8H), 3.22 (s, 3H), 2.91-2.85 (m, 4H), 2.65 (br, 2H), 2.55 (br, 2H), 2.11-2.0 (br, m, 8H), 1.33 (s, 12H), 0.64 (s, 6H); ¹³C NMR (CDCl₃, 125 MHz, 298 K): d 155.2, 150.9, 144.3, 141.8, 141.4, 135.3, 133.4, 132.6, 126.3, 125.6, 125.5, 124.4, 122.8, 84.0, 65.5, 52.4, 51.8, 51.7, 41.1, 29.9, 28.9, 28.2, 28.0, 25.1, 21.0, 20.9, 20.7, 1.2, 0.2, −0.7, −1.1.

### Example 19: Preparation of Compound Represented by Compound 20

A compound represented by Chemical Formula 20 (Compound 20) was prepared by a method of the following Reaction Formula 19.

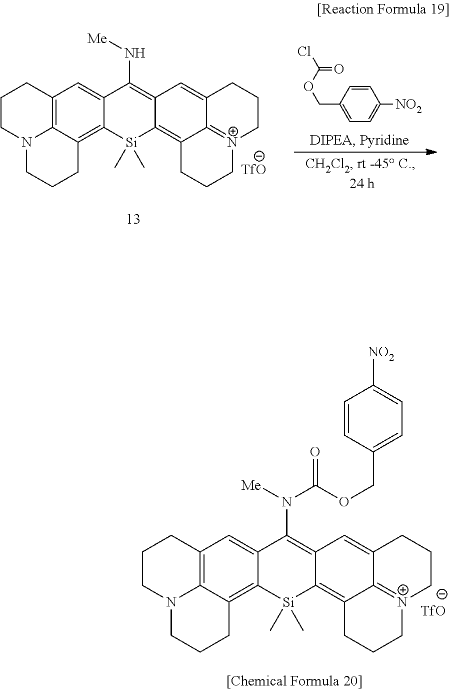

In order to prepare Compound 20, after a final product Compound 13 (30 mg, 0.05 mmol) obtained in the same manner as in Example 12 was dissolved using 5 mL of anhydrous dichloromethane, 44 μL, of 0.25 mmol diisopropylethylamine (DIPEA) and 40 μL, of 0.5 mmol pyridine were added thereto, the resulting solution was stirred at room temperature for 10 minutes, and then 108 mg of 0.5 mmol 4-nitrobenzylchloroformate was added thereto, and the resulting mixture was mixed by stirring at 45° C. for 24 hours. And then, the mixed solution was extracted using dichloromethane (2×10 mL) and 1 N hydrogen chloride, and the organic layer was dried with anhydrous sodium sulfate. And then, after the solvent was removed under a reduced pressure condition of 40 mbar, the residue was purified using column chromatography in which it passed through silica gel (Merck-Silicagel 60, 230-400 mesh), thereby obtaining a green solid compound. During the purification, 5% MeOH/CH₂Cl₂ was used as an eluent. And then, Compound 20 (5 mg, 12%) was finally obtained by again purifying the green solid compound through high performance liquid chromatography (HLPC). As a result of observing the prepared Compound 20, the NMR spectrum was confirmed as ¹H NMR (CD₃OD, 500 MHz, 298 K): d 8.32 (d, J=14.5 Hz, 2H), 7.30 (d, J=15 Hz, 2H), 7.09 (s, 2H), 5.18 (s, 2H), 3.62-3.55 (m, 8H), 3.24 (s, 3H), 2.98-2.94 (m, 4H), 2.77-2.66 (m, 2H), 2.63-2.53 (m, 2H), 2.09-1.93 (br, m, 8H), 0.67 (d, J=14.0 Hz, 6H).

### Example 20: Confirmation of Photophysical Properties of Amino-Si-Pyronin Compound

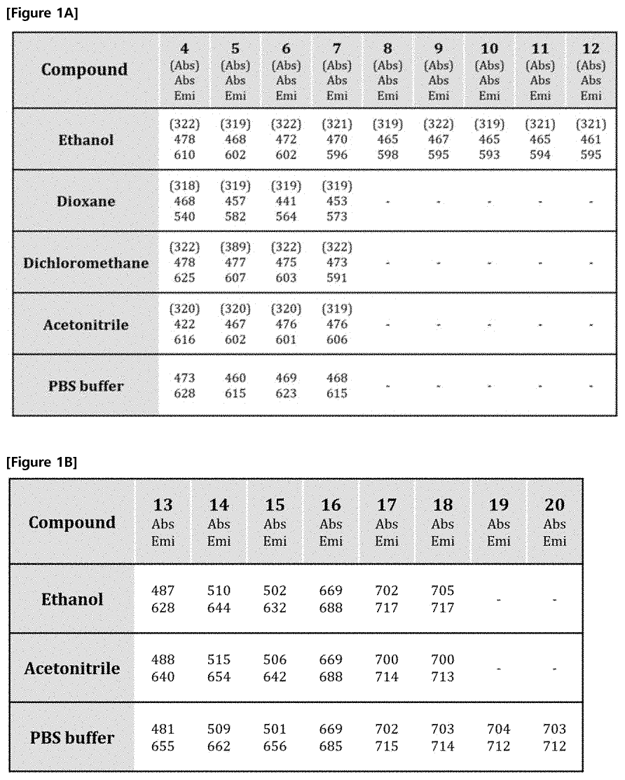

In order to confirm the absorption characteristics and fluorescence emission characteristics of Compounds 4 to 20 prepared in the same manner as in Examples 3 to 19, that is, amino-Si-pyronin compounds, ethanol, dioxane, dichloromethane, acetonitrile, and phosphate buffered saline including 1% DMSO, including any one of Compounds 4 to 12 at a concentration of 5 μM, were charged into a quartz cell having a passage length of 1 cm, the ultraviolet/visible (UV/Vis) absorption spectra were measured using an HP8453 ultraviolet/visible spectrophotometer, and the maximum absorption wavelength values are illustrated in FIG. 1A. Further, ethanol, acetonitrile, and phosphate buffered saline including 1% DMSO, including any one of Compounds 13 to 21 at a concentration of 10 μM, were charged into a quartz cell having a passage length of 1 cm, the ultraviolet/visible (UV/Vis) absorption spectra were measured in the same manner as described above, and the maximum absorption wavelength values are illustrated in FIG. 1B.

### Example 21: Confirmation of Microscopy Imaging Using Cells

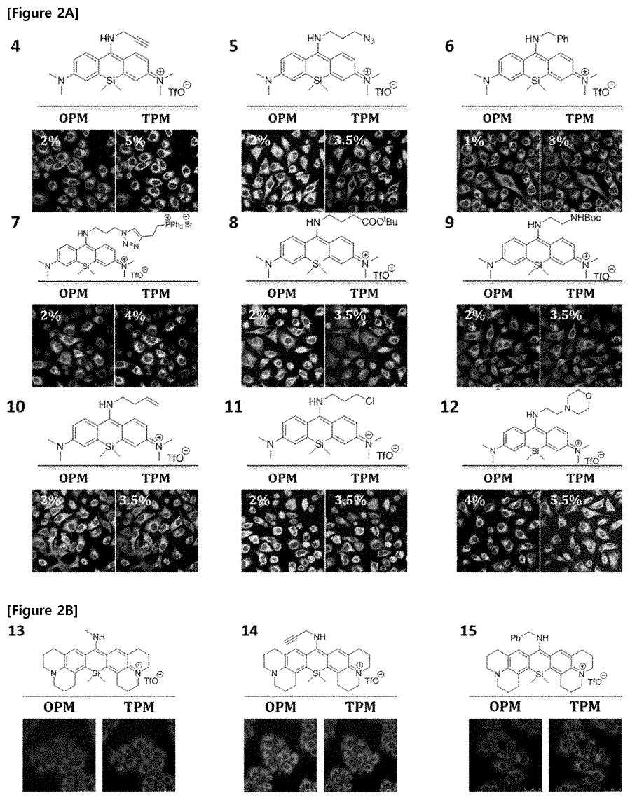

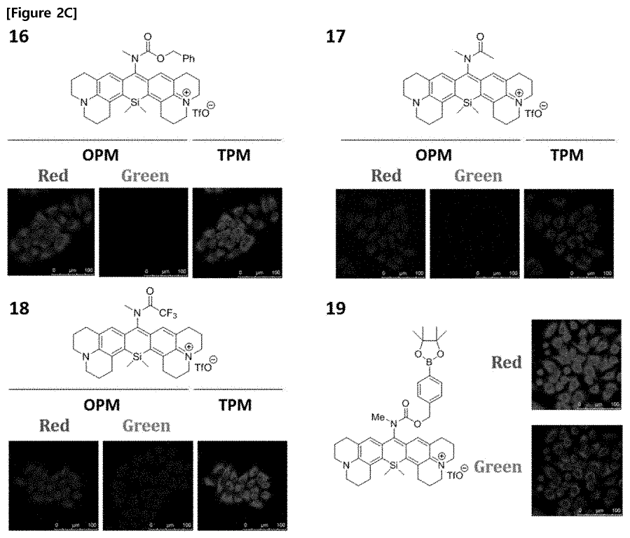

In order to confirm whether Compounds 4 to 12 prepared in the same manner as in Examples 3 to 11 could be used for imaging of cells, HeLa cells were treated with the compounds, and then images were obtained using a one- or two-photon microscope. More specifically, the HeLa cells were inoculated into Dulbecco's Modified Eagles Medium (DMEM) supplemented with 10% fetal bovine serum (FBS) and 1% antibiotic, and cultured at a density of 2×10⁶ cells in a 60 mm culture dish under conditions of 5% CO₂-95% air and 37° C. And then, after the cultured cells were respectively treated with 10 μM of Compounds 4 to 12 and allowed to react for 30 minutes, the culture solution was removed, and the culture solution was washed three times using phosphate buffered saline (PBS) to be completely removed. And then, the cells were fixed by adding a 4% formaldehyde solution thereto, and observed using a one-photon microscope or a two-photon microscope (TCS SP5 II, Leica, Germany). The results are illustrated in FIG. 2A.

As illustrated in FIG. 2A, it was confirmed that as a result of observing the cells treated with any one compound of Compounds 4 to 12 by a one-photon fluorescence microscope, fluorescence was observed in a channel of 498 to 800 nm at an excitation wavelength of 488 nm, and as a result of observing the cells treated with any one compound of Compounds 4 to 12 by a two-photon fluorescence microscope, fluorescence was observed in a blue channel (430 to 480 nm), a green channel (500 to 550 nm), a yellow channel (565 to 605 nm), and a red channel (625 to 675 nm) at an excitation wavelength of 900 nm.

### Example 22: Confirmation of Microscopy Imaging Using Cells

In order to confirm whether Compounds 13 to 19 prepared in the same manner as in Examples 12 to 18 could be used for imaging of cells, A549 cells were treated with the compounds, and then images were obtained using a one- or two-photon microscope. More specifically, the A549 cells were inoculated into Dulbecco's Modified Eagles Medium (DMEM) supplemented with 10% fetal bovine serum (FBS) and 1% antibiotic, and cultured at a density of 2×10⁶ cells in a 60 mm culture dish under conditions of 5% CO₂-95% air and 37° C. And then, after the cultured cells were respectively treated with 1 μM of Compounds 13 to 15 or 10 μM of Compounds 16 to 19 and allowed to react for 30 minutes, the culture solution was removed, and the culture solution was washed three times using phosphate buffered saline (PBS) to be completely removed. And then, the cells were fixed by adding a 4% formaldehyde solution thereto, and observed using a one-photon microscope or a two-photon microscope (TCS SP5 II, Leica, Germany). The results are illustrated in FIGS. 2B and 2C.

As illustrated in FIG. 2B, it was confirmed that as a result of observing the cells treated with any one compound of Compounds 13 to 15 by a one-photon fluorescence microscope, fluorescence was observed in a channel of 500 to 800 nm at an excitation wavelength of 488 nm, and as a result of observing the cells treated with any one compound of Compounds 13 to 15 by a two-photon fluorescence microscope, fluorescence was observed in a channel of 500 to 800 nm at an excitation wavelength of 900 nm.

Further, as illustrated in FIG. 2C, it was confirmed that as a result of observing the cells treated with any one compound of Compounds 16 to 19 by a one-photon fluorescence microscope, fluorescence was observed in a red channel (650 to 800 nm) and a green channel (500 to 630 nm) at an excitation wavelength of 488 nm and 633 nm, respectively, and it was confirmed that as a result of observing the cells treated with any one compound of Compounds 16 to 19 by a two-photon fluorescence microscope, fluorescence was observed in a yellow channel (565 to 605 nm) and a red channel (625 to 675 nm) at an excitation wavelength of 900 nm.

Through the results, it was confirmed that even in an experiment using cells, among various Compounds 4 to 20 based on the following Chemical Formula 1 or 3, all the compounds stably exhibited similar fluorescence emission characteristics in cells, and through this, it was confirmed not only that Compounds 4 to 20 of the present invention could be applied to bioimaging, but also that all the compounds of the present invention could be used as a one-photon and/or two-photon absorption fluorescent substance(s). Accordingly, it was confirmed in the present invention that various fluorescence emission compounds could be prepared by changing only an R group based on a structure of an amino Si-pyronin compound represented by the following Chemical Formula 1 or a julolidine-based amino-Si-pyronin compound represented by the following Chemical formula 3, and through this, it could be confirmed that it was possible to prepare fluorescent probes having various traits without limitation based on a structure of the following Chemical Formula 1 or 3.

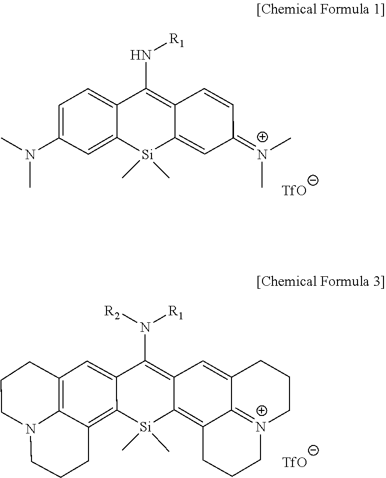

In addition, it was confirmed that in the case of Compound 16, fluorescent signals were observed in a red channel, and in the case of Compound 13, fluorescent signals were observed in a yellow channel Through this, it could be confirmed that because Compound 16 is a compound derived from Compound 13, two fluorescences could be simultaneously emitted through the Stokes shift when the form of Compound 13 in the form of a secondary amine was converted by an intramolecular exchange reaction. Accordingly, when one sensing system detects a substrate using the same, the compound can be applied to a ratiometric fluorescent probe in which the ratio is gradually changing depending on the concentration of the substrate, and through this, it could be confirmed that the compounds of the present invention could be used for ratiometric bioimaging. Furthermore, different fluorescences are observed depending on the change in each structure, so that it could be confirmed that the compounds of the present invention could be used to simultaneously detect various substrates, and fluorescent probes having more various traits could be prepared by partially changing structures of a compound represented by Chemical Formula 1 or 3.

The above-described description of the present invention is provided for illustrative purposes, and the person skilled in the art to which the present invention pertains will understand that the present invention can be easily modified into other specific forms without changing the technical spirit or essential features of the present invention. Therefore, it should be understood that the above-described Examples are only exemplary in all aspects and are not restrictive.

### INDUSTRIAL APPLICABILITY

An amino-Si-pyronin compound-based one- or two-photon absorption fluorescent substance of the present invention can be used to develop fluorescent probes having various characteristics on the basis of the fluorescent substance, can be used for imaging and diagnosing various diseases by using the fluorescent probe or binding a target for a marker of a specific disease to the fluorescent probe, and can be applied variously to analysis and imaging of specific materials in vivo.

1. An amino Si-pyronin compound represented by the following Chemical Formula 1 or pharmaceutically acceptable salt thereof: 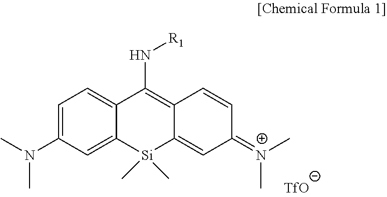
in the Chemical Formula 1,
R₁ is a substituted or unsubstituted alkyl group having 1 to 18 carbon atoms.

2. The compound or pharmaceutically acceptable salt thereof of claim 1, wherein the compound or pharmaceutically acceptable salt thereof is a one-photon absorption fluorescent substance or a two-photon absorption fluorescent substance.

3. The compound or pharmaceutically acceptable salt thereof of claim 1, wherein the compound or pharmaceutically acceptable salt thereof emits fluorescence in long wavelength regions or near-infrared regions of 625 nm or greater.

4. A julolidine-based amino-Si-pyronin compound represented by the following Chemical Formula 3 or pharmaceutically acceptable salt thereof: 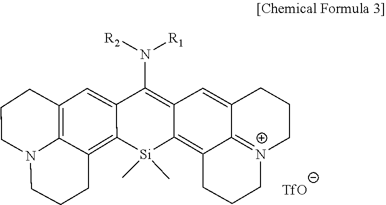
in the Chemical Formula 3,
R₁ is a substituted or unsubstituted alkyl group having 1 to 18 carbon atoms, and
R₂ is hydrogen, or a substituted or unsubstituted acyl group.

5. The compound or pharmaceutically acceptable salt thereof of claim 4, wherein the compound or pharmaceutically acceptable salt thereof is a one-photon absorption fluorescent substance or a two-photon absorption fluorescent substance.

6. The compound or pharmaceutically acceptable salt thereof of claim 4, wherein the compound or pharmaceutically acceptable salt thereof emits fluorescence in long wavelength regions or near-infrared regions of 625 nm or greater.

7. A cell- or tissue-imaging method, the method comprising treating cells or tissues with the compound or pharmaceutically acceptable salt thereof of claim 1 and observing the treated cells or tissues.

8. The method of claim 7, wherein the observing of the treated cells or tissues uses a one-photon fluorescence microscope or a two-photon fluorescence microscope.

9. A diagnostic composition, the composition comprising the compound or pharmaceutically acceptable salt thereof of claim 1 as an active ingredient.

10. A method for preparing an amino Si-pyronin compound represented by the following Chemical Formula 1, the method comprising: (a) preparing a mixture by adding trifluoromethanesulfonic anhydride to 3,7-bis(dimethylamino)-5,5-dimethyldibenzo[b,e]silin-10(5H)-one; and
(b) adding an amine to the mixture: 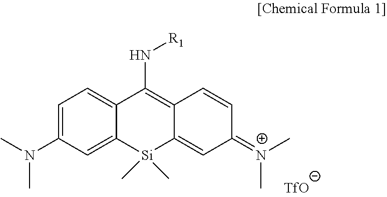
in the Chemical Formula 1,
R₁ is a substituted or unsubstituted alkyl group having 1 to 18 carbon atoms.

11. A method for preparing a julolidine-based amino-Si-pyronin represented by the following Chemical Formula 2, the method comprising:
(a) preparing a mixture by adding trifluoromethanesulfonic anhydride to a compound represented by the following Chemical Formula 22; and
(b) adding an amine to the mixture: 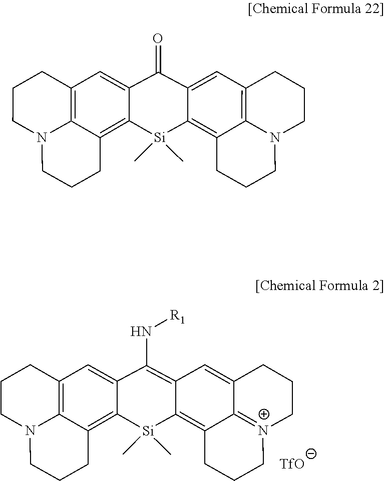
in Chemical Formula 2,
R₁ is a substituted or unsubstituted alkyl group having 1 to 18 carbon atoms.

12. A method for preparing a julolidine-based amino-Si-pyronin represented by the following Chemical Formula 3, the method comprising:
(a) preparing a mixture by adding diisopropylethylamine and pyridine to a compound represented by the following Chemical Formula 2; and
(b) adding a compound for an acylation reaction to the mixture: 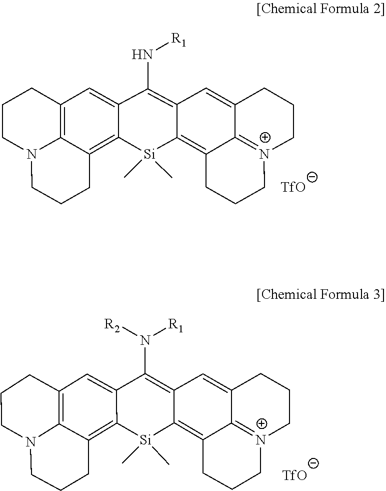
in the Chemical Formula 2 and 3,
R₁ is a substituted or unsubstituted alkyl group having 1 to 18 carbon atoms, and
in the Chemical Formula 3,
R₂ is a substituted or unsubstituted acyl group.

13. A method for obtaining a fluorescence image for diagnosis, the method comprising administering the compound or pharmaceutically acceptable salt thereof of claim 1 in vivo or to a tissue.

14. A use of the compound or pharmaceutically acceptable salt thereof of claim 1 for diagnosis.

15. A cell- or tissue-imaging method, the method comprising treating cells or tissues with the compound or pharmaceutically acceptable salt thereof of claim 4 and observing the treated cells or tissues.

16. The method of claim 15, wherein the observing of the treated cells or tissues uses a one-photon fluorescence microscope or a two-photon fluorescence microscope.

17. A diagnostic composition, the composition comprising the compound or pharmaceutically acceptable salt thereof of claim 4 as an active ingredient.

18. A method for obtaining a fluorescence image for diagnosis, the method comprising administering the compound or pharmaceutically acceptable salt thereof of claim 4 in vivo or to a tissue.

19. A use of the compound or pharmaceutically acceptable salt thereof of claim 4 for diagnosis.
